(19) United States
(12) Patent Application Publication
(10) Pub. No.: US 2024/0225114 A1
(43) Pub. Date: Jul. 11, 2024

(54) Electronic cigarette used in conjunction with mouth and nose

(71) Applicant: Qi ZHANG, Shenzhen (CN)

(72) Inventors: Qi ZHANG, Shenzhen (CN); Lin ZHANG, Suzhou (CN)

(21) Appl. No.: 18/611,086

(22) Filed: Mar. 20, 2024

Related U.S. Application Data
(63) Continuation-in-part of application No. PCT/CN2021/124562, filed on Oct. 19, 2021.

(30) Foreign Application Priority Data
Sep. 24, 2021 (CN) 202111118288.9

(51) Int. Cl. A24F 40/485; A24F 40/42; A24F 40/46; A24F 40/57; A24F 40/95

(52) U.S. Cl. CPC A24F 40/485; A24F 40/42; A24F 40/46; A24F 40/57; A24F 40/95

(57) ABSTRACT

An electronic cigarette used by cooperation of mouth and nose, including: a mouth inhalation module and a nasal inhalation module, the mouth inhalation module adopts one of a vapor electronic cigarette and a heat-not-burn electronic cigarette. The nasal inhalation module is arranged outside or inside of the mouth inhalation module, and the nasal inhalation module contains at least one nasal inhalation material carrier (1501). A nasal inhalation material is placed in the nasal inhalation material carrier (1501). The essence and nicotine can be partially or completely transferred from mouth inhalation module into nasal inhalation module of the e-cigarette. The nasal inhalation material is a combination of one or more of essence, alkaloid, nicotine salt, e-liquid, tobacco extracts, tobacco smoke condensate, cannabinoids, essence solvents, and the nasal inhalation material can be heated by the heating component that installed in the nasal inhalation module.

### CROSS REFERENCE

This application is a continuation in part of International (PCT) Patent Application No. PCT/CN2021/124562, filed on Oct. 19, 2021, which claims priority of Chinese Patent Application No. 202111118288.9, filed on Sep. 24, 2021, the entire contents of which are hereby incorporated by reference in their entirety.

### FIELD OF THE INVENTION

The present invention relates to the field of electronic cigarette, and in particular to an electronic cigarette used by cooperation of mouth and nose.

### Description of the Related Art

Electronic cigarettes, also known as electronic atomizers, are mainly divided into two categories: vapor electronic cigarette (Vapor) and heat-not-burn electronic cigarette (HNB).

Comparing with traditional flue-cured tobacco, these two categories of electronic cigarettes both can transmit nicotine to mouth, as well as greatly reduce the inhalation of tar, carbon monoxide, nitric oxide and other harmful substances into human body, whereby they have been used by hundreds of millions of users around the world as “harm reduction” products. Moreover, the atomizer and heater for herbs or cannabidiol (CBD) factions, such as lily, licorice, cannabidiol and cannabis polyphenols, which containing no any nicotine, are commonly classified as electronic cigarettes.

Up to now, the research on the harms of electronic cigarettes is not thorough enough, and new harms are ignored under the halo of “harm reduction”. For example, the essence in e-liquid will be inhaled into lungs by mouth with the atomized aerosol, and the essence preloaded within bead set in the filter tip as well as the essence in HNB solid cartridge or paste material will also be inhaled into lungs by mouth with the atomized aerosol or HNB smoke. Due to the physiological differences of different human organs, food essences which are safe to the stomach may not be equally safe to the lungs. For example, Vitamin E Acetate is a food additive that has been used by people for a long time, however, after Vitamin E Acetate being inhaled into lungs with electronic cigarette aerosol, it will not be digested and metabolized, but covers the alveoli, causing inflammation of the lungs and even human body death; butanedione and 2,3-pentanedione have been allowed adding to food as food essences for a long time, but they will be deposited in the trachea after being inhaled into lungs, causing obstruction, worsening respiratory infections and leading to “popcorn lungs”.

In addition, grease, fructose, sucrose, acesulfame sweetener, aspartame sweetener, vitamins, long-chain esters, ethylene glycol, borax, coumarin, (4-ethoxyphenyl)urea, cinnamyl anthranilate, safrole, thiourea, acetoin, and the likes, are all conventional ingredients in edible essences, but once they are inhaled into lungs with the electronic cigarette aerosol or HNB smoke, they will seriously harm the user's health.

Nicotine/nicotine salt, propylene glycol, glycerol, and essence are the four major components of e-liquid. Sweeteners, cooling agents, and aroma substances are used to be classified as essence by people. Essences such as sweeteners, cooling agents, and aroma substances are also required adding to the solid cartridge or paste material of HNB e-cigarettes.

E-cigarettes containing essences are called as flavored e-cigarettes or aroma e-cigarettes. Today, essence is an indispensable key component for both vapor e-cigarettes and HNB e-cigarettes. E-cigarettes lacking essences will lose the competitiveness and cause users to choose traditional flue-cured tobacco again, which do harm to users more seriously. However, the types of essences are very numerous and jumbled. These essences include natural, synthetic, or animal extracts and plant extracts. Along with essences, there are also essence solvents, as well as by-products in the essence production process and uncertain impurities mixed in the essence filling process, and so on.

In addition, propylene glycol and glycerol are conventional essence solvents in e-liquid, and these two accounts for about 90% by mass in e-liquid. Wherein, propylene glycol in e-cigarettes is usually derived from chemical industries such as coal and petroleum rather than plant extraction, and scientific research has confirmed that propylene glycol causing serious harm to the mouth cavity of e-cigarette users. Although glycerol is said deriving from plants, there is still in the absence of scientific and systematic research of pathology and toxicity to determine whether it is still safe after entering into the lungs and depositing in the alveoli for a long term.

At present, more and more essences, essence solvents and essence-related substances in e-liquid and HNB solid cartridge or paste material are inhaled into the lungs from mouth, and people can not conduct the pathological, toxicological research and scientific judgment of their hazards in time, thereby causing a lag in the safety precautions. For example, both Vitamin E Acetate and butanedione caused deaths in 2019 till people discovered their harm to lungs and began to ban them in e-cigarettes. However, the essence solvents and a large number of essences that having not been scientifically determined to be safe for lungs are still being used in the e-cigarette industry, which undoubtedly increases the risk for users.

The atomizing temperature of e-liquid of vapor e-cigarette is about 180-280° C., and the heating temperature of HNB e-cigarette solid cartridge or paste material is about 250-380° C., wherein the essences, essence solvents and essence-related substances within these cigarette materials have a risk of decomposing into formaldehyde, acrolein, acetone, and other hazardous substances at these high temperatures.

Currently, users employ e-cigarettes by two inhaling methods: “small circulation through mouth” and “large circulation through lungs”. Specifically, the essence molecules within the atomized aerosol or HNB smoke inhaled by mouth go through the small circulation of “essence→mouth mucosa” or the large circulation of “essence→mouth mucosa→throat→trachea→bronchus→lungs→bronchus→trachea→throat→mouth mucosa”, and being exhaled out to air from the nose and or mouth, and till then the essence can be sensed by the olfactory nerves in the nasal cavity. However, not only is there a loss of the number of essence molecules, but also they will be diluted and interfered by the aerosol or other substances in the surroundings, resulting in users complaining that the e-cigarettes are poorly flavors reproduction level, which in turn cause manufacturers adding more essences in the e-liquid and HNB solid cartridge or paste material.

Furtherly, a major problem is that even if each mouthful of vapor aerosol or HNB smoke from e-cigarette and each mouthful smoke from traditional flue-cured tobacco contain almost the same amount of nicotine, but the nicotine satisfaction of e-cigarettes is still worse than that of traditional flue-cured tobacco, and it is difficult to simulate the wonderful flavor of traditional flue-cured tobacco even lots of tobacco essences are added to e-cigarettes. This problem causes many tobacco users dislike e-cigarettes. People try to add tobacco extract paste or tobacco smoke condensate in e-liquid as a special tobacco essence, but both these two tobacco essences are high opaque and viscous even after being diluted with lots of essence solvents, and they will block the atomizing core and cause charring. When adding these two tobacco essences to HNB solid cartridge or paste material, the using experience is still worse than that of traditional flue-cured tobacco. Patent No. CN103271449A disclosed a snuff-type electronic cigarette, which is a single-channel method to inhale smoke by nose replacing mouth, completely abandoning mouth as an inhalation channel of e-cigarettes. The similar patents further include CN211065041U and CN205161890U.

WO2011109848A1 disclosed an inhaler component, as claimed that “but the flavor is essentially nicotine-free”, whereby the user's nasal cavity can not directly inhale nicotine by this inhaler component. Moreover, there is no heating component in the flavor reservoir (6), resulting in poor volatility of flavor (16) especially in low temperature environments, where the user's olfactory perception for the flavor (16) will seriously decrease. Improvement is needed.

### Contents of the Invention

The main technical problem to be solved by the present invention is: to improve the using experience of e-cigarettes close to that of traditional flue-cured tobacco, and to reduce the essences, essence solvents, and essence-related substances entering the user's lungs.

In order to solve or partially solve the above problems, the present invention adopts a technical solution: to provide an e-cigarette used by cooperation of mouth and nose, including: a mouth inhalation module and a nasal inhalation module, the mouth inhalation module adopts one of a vapor e-cigarette and a heat-not-burn e-cigarette, the nasal inhalation module is arranged outside or inside of the mouth inhalation module, and the nasal inhalation module contains at least one nasal inhalation material carrier, and nasal inhalation material is placed in the nasal inhalation material carrier. The nasal inhalation material is a combination of one or more of essence, nicotine or other alkaloid, nicotine salt, e-liquid, tobacco extract paste, tobacco extract liquid, tobacco smoke condensate, cannabinoids, essence solvents, but the nasal inhalation material does not include air, and the nasal inhalation module further contains the component that can heat the nasal inhalation material.

The e-cigarette still retains the mouth transmission channel by the mouth inhalation module to inhale the atomized aerosol of e-liquid or the smoke of HNB solid cartridge and paste material, and the additional nasal inhalation module is used as the transmission channel for the volatiles of nasal inhalation material to reach the user's nose directly, whereby the e-cigarette being used for inhalation by the dual channels of mouth and nose at the same time. The essences and nicotine previously added to the vapor e-liquid and HNB solid cartridge or paste material in the mouth inhalation module are partially transferred or completely transferred into the nasal inhalation module, and the nasal inhalation material in the nasal inhalation module release volatiles at room temperature as well as being slightly heated and or fan blown, whereby the volatiles of nasal inhalation material in the nasal inhalation module can be directly transmitted to the user's nasal cavity without entering the mouth cavity.

In a preferred embodiment of the present invention, the vapor e-cigarette adopts one of a high-power e-cigarette, a low-power e-cigarette, a CBD factions electronic cigarette, an open type e-cigarette, a closed type e-cigarette, an ultrasonic atomized e-cigarette, a disposable vapor e-cigarette, a cartridge-changing vapor e-cigarette, and the heat-not-burn e-cigarette adopts one of a HNB e-cigarette that consuming solid cartridge and a HNB e-cigarette that consuming paste material.

So far the above-mentioned vapor e-cigarettes and heat-not-burn e-cigarettes of the prior art have only a mouth inhalation module, and components of the mouth inhalation module can be categorized into four sub-modules: an inhalation nozzle module, an atomizing or heating module, a circuit module and a display module, wherein:

The inhalation nozzle module contains components such as nozzles or filters tip, and the likes; The atomizing or heating module is further classified into: (1) e-liquid atomization module, containing empty atomizing cartridge, e-liquid storage tank, e-liquid, cotton atomizing core or ceramic atomizing core, aerosol channel, the atomizing cartridge base with magnetic column and electrode tip, and other components, (2) ultrasonic atomization module or piezoelectric ceramic atomization module, (3) HNB resistance heating module, containing the heater of heating element, heating rod, heating tube, heating pot, heater base with an electrical connection port, cartridge, cartridge sleeve, cartridge base and other components, (4) other types heating modules, such as electromagnetic heating, microwave heating, infrared heating, and so on, with corresponding components respectively;

The circuit module contains components such as printed circuit board assembly (PCBA), temperature coefficient of resistance (TCR) thermostat, starter, temperature sensor, indicator light, switch, gear controller, battery, wire interface, wires, and the likes;

The display module contains components such as data collector and display screen, and the likes. The display module is not a necessary module for e-cigarette, but some control components can further integrated into the display module, such as control components that can adjust the output power.

The present invention provides a nasal inhalation module on the basis of the mouth inhalation module of e-cigarette, and the nasal inhalation module may or may not be connected with the circuit module and the display module in the above mentioned four sub-modules in the mouth inhalation module.

In a preferred embodiment of the present invention, the essence includes but not limited to one or more of fruit peel, fruit, fruit essence, cooling agent essence, sweetener essence, tobacco essence, meat essence, wine essence, food essence, plant herbal essence, animal spice essence, biosynthetic essence and microbial fermentation essence. In addition, the essence further includes some substances without characteristic aroma such as flavonoids, polyphenols and so on, as well as including some substances without easily perceived aroma. Specifically, the cooling agent essence such as menthol essence, WS-23 (N-ethyl-L-menthyl formamide) essence, WS-3 (peppermint amide) essence, and the likes; the sweetener essence such as sucralose essence, neotame essence, and so on; the fruit essence such as linalool essence, β-damascenone essence, passion fruit essence, and so on; the tobacco essence such as tobacco extracts, blends of several monomer essences, and the likes; the meat essence such as animal fats, artificial ethyl maltol, and the likes; the food essence such as mung bean essence, and the likes; the wine essence such as ethyl caproate, and the likes; the plant herbal essence such as russianolive flower essence, rose essence or flos sophorae essence, and the likes; the animal spice essence such as musk, civet, castoreum, ambergris, and the likes; the biosynthetic essence such as vanillin and 2-phenylethanol synthesized by biotransformation, and the likes; the microbial fermentation essence such as acid essence, alcohol essence, ketone essence, lactone essence, and the likes. A small amount of solvent coming with the essence as an essence solvent, such as water, propylene glycol, medium-chain triglycerides, caprylic triglycerides, and so on, belongs to nasal inhalation material together with the essence.

In a preferred embodiment of the present invention, it further includes a component retainer for fixing the nasal inhalation module on the outside or inside of the mouth inhalation module, wherein the component retainer includes but not limited to one or more of double-sided adhesive tape, gummed tape, glue, string, clamp, snap hook, magnet, screw, slot, jack and plug, and fixing ring, as well as some machine parts of bolt fixation, pin shaft fixation, and sleeve fixation.

In a preferred embodiment of the present invention, the nasal inhalation material carrier is a combination of one or more of bottle, pot, electric heating plate and electric heating pot. The electric heating plate adopts a miniature mesh electric heating plate or a non-mesh electric heating plate with integrated resistive heating component. In addition to resistance heating, the heating method in the nasal inhalation module can be one of electromagnetic heating, infrared heating, microwave heating, which usually need battery providing electrical energy for the heating. The heating component can be a combination of one or more of battery, circuit switch, controller, sensor, wires, DC/AC converter, heater, printed circuit board, microprocessor device, LC circuit, inductor, microwave generator, infrared generator, heating indicator light, charging indicator light and charging port cover and so on. Moreover, the volatiles of nasal inhalation material also can be pushed out with a little temperature rising by some ultrasonic element, then the ultrasonic element belongs to the heating component in the nasal inhalation module. Moreover, the nasal inhalation material can be heated by some non-electrical energy and without using battery, for example, chemical exothermic reactants, high-temperature objects inside or beside, mechanical friction heating, which also belongs to the component that can heat the nasal inhalation material in the nasal inhalation module.

In addition, the height of the bottle cap, pot spout plug and electric heating pot sealing cover that accompanying the nasal inhalation material carriers may be increased appropriately, so as to open or close these nasal inhalation material carriers from the corresponding cover hole outside of the chamber cover.

In addition, a push-type switch is set over the chamber cover so as to control the volatiles outlet valve of the nasal inhalation material pot airtight cover, and the user can directly control the release of the nasal inhalation material volatiles from outside of the nasal inhalation material pot without opening the chamber cover to remove the nasal inhalation material pot airtight cover.

In a preferred embodiment of the present invention, a first battery is arranged in the mouth inhalation module, and the nasal inhalation module can share the first battery with the mouth inhalation module, or the nasal inhalation module may contain a second batter, or the nasal inhalation module may be equipped without battery. The second battery may be a rechargeable battery or a disposable battery, and when the second battery is a rechargeable battery, a corresponding charging port needs to be provided on the e-cigarette.

In a preferred embodiment of the present invention, the nasal inhalation module further contains an electric heater controller, and the first battery or the second battery is connected with the electric heater controller for power supply, and the electric heater controller is connected with the heating component in the nasal inhalation module for heating control.

In a preferred embodiment of the present invention, the nasal inhalation module further contains a fan and a fan controller corresponding to the nasal inhalation material, and the first battery or the second battery is connected with the fan controller for power supply, and the fan controller is connected with the fan for operation control. The electric heater controller and the fan controller can be integrated on the electric circuit board of the mouth inhalation module or the nasal inhalation module, or respectively adopts conventional electrical switch such as miniature knob switch, push-type switch, inductive switch and the likes, which can be used to control both the operation and stopping of these electrical appliances, and to adjust their operating gears. In order to facilitate the user to operate from the outside of the e-cigarette rod, these controllers can be completely or partially installed on the chamber cover through the alignment holes.

In addition, in a few special cases, in addition to connecting with the fan and the electric heater in the nasal inhalation module, the second battery may further connect with some electrical appliances and electronic components of the mouth inhalation module, for example PCBA, starter or sensor, and the likes, and when the second battery connects with all electrical appliances and electronic components in the mouth inhalation module, the second battery is equivalent to the first battery.

In a preferred embodiment of the present invention, the nasal inhalation module is arranged on the shell of the mouth inhalation module by the component retainer.

In a preferred embodiment of the present invention, a chamber that corresponding to the nasal inhalation module is provided in the shell of the mouth inhalation module, and the nasal inhalation module is partially or completely implanted into the chamber:

Wherein, the nasal inhalation module can be arranged inside of the e-cigarette rod closing to the cartridge in the mouth inhalation module. In prior art, in order to connect the cartridge of the vapor e-cigarette or the cartridge of HNB e-cigarette, a chamber is provided in head of the e-cigarette rod to facilitate the insertion of the cartridge, but there is no remaining space in the chamber after the cartridge being inserted. In order to implant the nasal inhalation module, it is necessary to extend the shell of the e-cigarette rod in the direction of the cartridge and lengthen the connection chamber by 5-200 mm, and then move the vapor e-cigarette base with magnetic columns and electrode tips forward accordingly or move the HNB e-cigarette base with heating element and wires forward accordingly thereby creating a chamber space for the nasal inhalation module with a length of 5-200 mm and almost the same height and width as those of the e-cigarette rod. It can be operated from the horizontal direction of the e-cigarette rod to sequentially implant and fix components of the nasal inhalation module such as nasal inhalation material carrier, fan, electric heater, battery and the likes into the chamber, and the alignment holes for the controllers of such as the fan and the electric heater are set in the chamber shell to facilitate control from the outside. By another method, a chamber cover can further be made on the shell of e-cigarette rod, operating from the vertical direction of the e-cigarette rod to sequentially implant and fix components of the nasal inhalation module such as nasal inhalation material carrier, fan, electric heater, battery and the likes into the chamber, and the alignment cover holes for the controllers of such as the fan and the electric heater are arranged on the chamber cover to facilitate control from the outside, and the chamber cover can seal the chamber through slot, so as to maintain a consistent appearance of the outer shell of the e-cigarette rod.

Wherein, the nasal inhalation module may also be set in the middle of the e-cigarette rod. Firstly, the ordinary vapor e-cigarette rod and HNB e-cigarette rod are lengthened by 5-200 mm, and then the corresponding components of the original atomizing or heating module, the circuit module and the display module are adjusted to be close to the two ends of the e-cigarette rod, and a chamber for the nasal inhalation module with a length of 5-200 mm and almost the same height and width as those of the e-cigarette rod is created in the middle of the e-cigarette rod. The adjustment of the position of the internal components of the e-cigarette rod is a task that can be carried out by those skilled in the art without creative effort. After opening the chamber cover, operating from the vertical direction of the e-cigarette rod to implant and fix the nasal inhalation material carrier, fan, electric heater, battery and other components of the nasal inhalation module into the chamber in sequence, and the alignment cover holes for the controllers of such as the fan and the electric heater are provided on the chamber cover to facilitate control from the outside.

Wherein, the fan in the nasal inhalation module can be a miniature floor-standing electric fan with an integrated electric motor, or a disc-type or desktop electric fan, and so on.

In a preferred embodiment of the present invention, the nasal inhalation module further contains a volatiles conduit, whereby the nasal inhalation module can only partially implanted into the above chamber. The volatiles conduit may further connect a volatiles conduit interface and an conduit extension tube or the likes to be a volatiles conduit assembly. One end of the volatiles conduit is close to the nasal inhalation material carrier or directly or indirectly connected with the nasal inhalation material carrier, and the other end of the volatiles conduit is close to the user's nasal cavity which may even be a hood covering the nose or hollow plugs inserted into the user's nostrils and the other end of the volatiles conduit may be shared with other people, wherein the outlets of the volatiles conduit to the nasal cavity may be one, two or more.

Components of the volatiles conduit assembly are connected or disassembled through corresponding threads, sockets and other structures, and the volatiles conduit may be a stereotyped single tube, a plug-in multi-inhalation tube, a telescopic sleeve type tube or a flexible universal tube, and the volatiles conduit can further be made with the appearance of elephant nose, cartoon nose, animal, plant, round hole, square, doll and trademark, and the likes, to increase the aesthetics of the volatiles conduit as well as the e-cigarette.

The technical principle of the present invention is: both mouth and nose are the important inhalation and exhalation channels of the human body, which can work independently or cooperatively, and the cooperative work can be performed simultaneously and synchronously. Essences are mostly organic small molecules with molecular weights less than 300, such as hydrocarbons, alkenes, alcohols, aldehydes, ethers, ketones, acids, phenols, esters, terpenes, sulfur-containing compounds, nitrogen-containing compounds, and the likes, which are easy to volatilize naturally as smells. Both heating and blowing can improve the volatilization speed of essence, nicotine or other alkaloids, nicotine salt, e-liquid, tobacco extract paste, tobacco extract liquid, tobacco smoke condensate, cannabinoids, solvents and other nasal inhalation materials as volatiles especially in a low temperature environment, whereby the volatiles of nasal inhalation material can be quickly perceived by the nasal cavity. According to some research: the human body's olfactory nerves to smells are mainly concentrated in the nasal cavity. The nasal cavity is a part of the skull, with a length of about 12-15 cm and a volume of about 9-16 ml in the human body. The total area of the nasal mucosa is about 150-180 cm². The blood vessels in the nasal mucosa are very numerous, with arteries, veins, and capillaries interwoven into a network. In addition, the ophthalmic and maxillary branches of the trigeminal nerve, the olfactory nerve, and the facial nerve branches, which are three out of the 12 pairs of brain nerves in the human body, are distributed in the nasal mucosa. The olfactory nerve in the nasal mucosa passes through the sieve hole and directly connecting to the olfactory bulb of the brain, while the nasal endothelial nerve bundle connects to the cerebrospinal fluid. The human body's olfactory perception mechanism can be briefly described as follows: the smell is identified by the “receptor” of the epithelial tissue of the nasal cavity and generates a “mirror image” nerve signal transmitting to the brain. The “mirror image” nerve signals generated from smells of different volatiles are differentiated, so the brain can identify specific volatiles. The olfactory system is characterized by its own nerves directly connecting to the brain, no need to go through the transduction to the central nervous system and then to the brain, so, “smells→nasal→brain” is the fastest olfactory identification channel by the unique directly connection. Similarly, the transmission path of nicotine through the nasal inhalation module is “nicotine→nasal→brain”. Nicotine signals are transferred to the brain through the nasal mucosa by three main pathways: the olfactory nerve pathway, the olfactory mucosal epithelial pathway, and the blood circulation pathway. Wherein, the blood circulation pathway is similar to the nicotine transferring path by mouth inhalation, which requires nicotine to be transferred into the brain blood first and then break through the blood-brain-barrier before the nicotine signal entering the perceptual area of the brain. But by the olfactory nerve pathway and the olfactory mucosal epithelial pathway, nicotine signals can be directly transmitted to the brain and enter the central nervous system, producing reflexive regulation of brain and visceral organs, thereby quickly achieving a sense of nicotine satisfaction. It can be seen that comparing to the intake of nicotine through the mouth inhalation module, the intake of nicotine through the nasal inhalation module has the advantages of a shorter and faster transmission path, as well as less nicotine loss during the transmission process. The present patent provides a nasal inhalation module on the e-cigarette, so that while the atomized aerosol or HNB smoke generated from the mouth inhalation module of e-cigarette are inhaled by the mouth channel, at the same time, the user's nasal channel directly inhales the volatiles of nasal inhalation material released from the nasal inhalation module. The heating component and fan blowing in the nasal inhalation module can improve the transmission efficiency of volatiles of nasal inhalation materials to the nasal cavity especially in low temperature environments. Moreover, there are few interfering substances when the volatiles of nasal inhalation material enter the nasal cavity from the nasal inhalation module, wherein the essence aroma to be accurately identified by the olfactory nerve, as well as efficient transportation of nicotine and other substances, thereby improving the flavor reproduction level as well as the using experience of e-cigarettes. In addition, the human nose does not have the swallowing function as that of the mouth. The volatiles of nasal inhalation material, for example, essence, essence-related substances, nicotine salt, tobacco extracts, tobacco smoke condensate and essence solvents will be blocked by the nasal hairs and nasal mucosa after entering the nasal cavity, and it is difficult for them to enter the user's lungs, thereby improving the using safety of e-cigarettes.

The essence and other nasal inhalation material in the nasal inhalation module can volatilize naturally at room temperature, as well as quickly volatilize by slightly heating or fan blowing whereby the essence and other nasal inhalation material can be perceived by the nasal olfaction in time especially in low temperature environments. The slightly heating temperature is usually 40° C.-170° C., further 45° C.-60° C., which is much lower than the atomization temperature of e-liquid and the heating temperature of HNB solid cartridge or paste material. Inhaling the essence aroma by the nasal inhalation module at low temperature can not only maintain the physiological activity of essence molecules, but also reduce the risk of decomposition to formaldehyde and other harmful substances from the high temperature heating of essences, essence solvents and essence-related substances.

To the inventor's surprise: after the essence and nicotine are partially transferred or completely transferred from the mouth inhalation module into the nasal inhalation module of e-cigarette, the flavor reproduction level of the e-cigarette will be greatly improved. The reason is analyzed that: essence and nicotine are partially or completely transferred from the mouth inhalation module into the nasal inhalation module of the e-cigarettes, which can be directly transmitted to the nasal cavity and brain, without the dilution and interception of human organs undergoing “small circulation through mouth” or “large circulation through lungs” by the mouth inhalation module.

To the inventor's surprise: some e-liquids become clearer and more transparent than before after the essence being partially transferred or completely transferred from the mouth inhalation module into the nasal inhalation module. The reason is analyzed that: many essences in e-liquid are derived from plant pressing or organic solvent extraction, which contain terpene essences. Although the terpene essences aroma are strong and distinctive to enhance the flavor characteristics of the vapor e-cigarettes, the solubilities of these terpene essence molecules in e-liquid are very poor, and they are easy to be separated out and cause the e-liquid turbid or even layered slicks and other serious appearance problems, compelling people to add more solvents or other new solvents to dissolve or dilute terpene essences in such type e-liquid, which not only reduce the concentration of essences aroma, but also increase the risk of lungs for these essence solvents. In the present invention, those insoluble essences can be by partially or completely transferred into the nasal inhalation module, whereby it not only reduces the risk of the essences and essence solvents entering the lungs from mouth, but also improving the appearance of the e-liquid by making it clearer and more transparent than before.

To the inventor's surprise: after the essence being partially transferred or completely transferred from the mouth inhalation module into the nasal inhalation module, leading to the decrease of the essence-related substances that directly contacted with the vapor atomizing core or HNB heating element at high temperature, and the troubles of the burnt atomizing core and the carbon deposition on atomizing core or on heating element, which have plagued the users of e-cigarette for a long time, are significantly alleviated.

To the inventor's surprise: after the essence being partially transferred or completely transferred from the mouth inhalation module into the nasal inhalation module of e-cigarette, due to the reduction of the risk of essence-related substances entering the lungs from mouth, the types of essences available for e-cigarette can be increased through the nasal inhalation module. For example, meat essences, wine essences and the likes, and even tobacco extract paste and tobacco smoke condensate can be used in nasal inhalation module, which are difficult to be used for the e-liquid in prior arts of vapor e-cigarettes due to the problems such as muddy, untransparent, viscous and easy blocking atomizing core. In addition, transferring out the essences from the mouth inhalation module of e-cigarettes may also simplify the essences modulation technology, as well as simplifying the production processes of mouth inhalation materials such as e-liquids, solid cartridges and paste materials.

To the inventor's surprise: after the nicotine being partially transferred or completely transferred from the mouth inhalation module into the nasal inhalation module of e-cigarette, not only the obtaining of nicotine satisfaction is faster than before, but less amount of nicotine is needed for the nicotine satisfaction each time, which will help users to slow down the nicotine addiction by e-cigarette. The detail reasons are as follows:

After being inhaled by mouth, the nicotine transmission pathway of the “small circulation through mouth” is “nicotine→mouth mucosa” and the “large circulation through lungs” is “nicotine→mouth mucosa→throat→trachea→bronchus→lungs→alveoli→brain blood→blood-brain-barrier→brain nervous system”, respectively. The transmission pathways are long for passing many intermediate organs, so nicotine will inevitably be intercepted by the intermediate organs during the step-by-step transmission process. Some of the intercepted nicotine will be absorbed and metabolized by the cellular tissues or blood of the intermediate organs, resulting in nicotine loss in the transmission pathway. Although others of the intercepted nicotine may be slowly transported to the brain blood through human cells or vascular circulation systems, which will be delayed to break through the blood-brain-barrier and integrate signal into the brain nervous system. Moreover, most of the residual nicotine, exhaled from the mouth and nose after passing through the “small circulation through mouth” or “large circulation through lungs”, will lost into the air, and only a little residual nicotine can be inhaled back into the nasal cavity, resulting in almost no nicotine signal directly transferring to the brain from the nasal cavity when using e-cigarettes singly by mouth. Therefore, excessive nicotine is needed to obtain the nicotine satisfaction each time because of the lost nicotine and delayed nicotine during the transmission process of mouth inhalation. Only by inhaling excessive nicotine, offsetting the lost nicotine and delayed nicotine, can the accumulation of nicotine in the human blood be sufficient and the nicotine signal in the brain reach the high peak as the important indicators of nicotine satisfaction. Therefore, it can be seen that when nicotine is inhaled by mouth alone, the transmission efficiency and utilization of nicotine are very low, leading to users frequently inhaling excessive amounts of nicotine each time.

In the present invention, the nicotine is partially or completely transferred from the mouth inhalation module into the nasal inhalation module of the e-cigarette, which can be directly inhaled into the user's nasal cavity through the nasal inhalation module and quickly transfer signal to the brain and obtain the nicotine satisfaction. Comparing to the e-cigarettes of prior art that nicotine singly inhaled by mouth, the transmission efficiency and utilization rate of the nicotine inhaled by cooperation of mouth and nose in the present invention are higher. Similar as stop eating after a person is full, when the nicotine signal and dopamine secretion reach the peak value in the brain with the nicotine satisfaction, then the user will stop inhaling nicotine. Therefore, the e-cigarette used by cooperation of mouth and nose in the present invention can help users to reduce the nicotine intake each time for the nicotine satisfaction.

The differences between the present invention and the prior arts of e-cigarette and snuff are as follows:

(1) In the e-cigarettes of prior art, both the nicotine and essence are heated up to 180-280° C. with the e-liquid of vapor e-cigarettes, or are heated up to 250-380° C. with the HNB solid cartridge or paste material, respectively, till then they can be inhaled into mouth. Some essences will lose biological activity and even decompose into formaldehyde, acrolein, acetone and other hazards at these high temperatures. In the present invention, the nasal inhalation materials such as essence and nicotine can not only volatilize naturally into the user's nasal cavity, but also enter the user's nasal cavity by the fan blowing or slightly heating by the heating component in the nasal inhalation module, specially improving the transmission efficiency of nasal inhalation materials to the nasal cavity when the user in a low temperature environment, wherein both essence and nicotine can be perceived by the nasal cavity in time. Moreover, the temperature of the slight heating is usually low, which can maintain the biological activity of most essence molecules without decomposition into hazardous substances.

(2) In the prior art, both the nicotine and essence are inhaled by mouth with the atomized aerosol of vapor e-cigarettes or with the smoke of HNB cartridge, after the dilution and depletion by many intermediate organs through the “small circulation through mouth” or “large circulation through lungs” and exhaled out to air from the nose or mouth, till then, some of the residual essence can be perceived by the olfactory nerve in the nasal cavity, resulting in a very low level of flavor reproduction. It also leads to a low nicotine satisfaction efficiency because of the lost nicotine and delayed nicotine in the transmission pathway, resulting in users having to inhale excessive amounts of nicotine each time for nicotine satisfaction. In the present invention, the essence and nicotine are partially or completely transferred from the mouth inhalation module into the nasal inhalation module of electronic cigarettes, the smell of essence will be directly recognized by the “receptor” of nasal epithelial tissue and generate “mirror image” nerve signals transmitting to the brain, whereby improving the flavor reproduction level of the e-cigarette, and nicotine can be directly inhaled by the user's nasal cavity to quickly transmit signals to the brain and obtaining nicotine satisfaction, whereby improving the transmission efficiency and utilization rate of nicotine, and helping users to further decrease the nicotine intake each time for nicotine satisfaction.

(3) Users inhale atomized aerosols or HNB smoke only through the mouth in the prior art of e-cigarettes, and users suck snuff powder only through the nose in the prior art of snuff, both are single inhalation channel. However, the present invention discloses an e-cigarette with dual inhalation channels that can be used by cooperation of mouth and nose simultaneously.

(4) In the prior art of snuff, the snuff powder is directly sucked into the nasal cavity. However, the snuff powder is usually a solid mixture of lime powder and crushed tobacco, wherein nicotine has a high concentration in some position of nasal cavity, which are harmful to the nasal mucosa and olfactory nerves. However, in the present invention, it is volatiles of the essence, nicotine and other nasal inhalation material which are uniformly concentrated and comfortable that directly inhaled into the nasal cavity by natural volatilization, slight heating or fan blowing from the nasal inhalation module of e-cigarette, which has minimal harm to the nose.

The mouth inhalation channel is completely abandoned in the patents of CN103271449A, CN211065041U and CN205161890U.

The flavor (16) claimed in the WO2011109848A1 is “but the flavor is essentially nicotine-free”. So, this inhaler component can not improve the transmission efficiency and utilization of nicotine, and there is no heating component in the flavor reservoir (6), resulting in poor volatility of the flavor (16) in low temperature environments, wherein the user's olfactory perception of flavor (16) will be seriously decreased.

Traditional flue-cured tobacco releases both mainstream smoke and sidestream smoke due to the heating function by burning or semi burning, wherein the mainstream smoke is inhaled by mouth and the sidestream smoke is inhaled by nose respectively. The flavor reservoir (6) in WO2011109848A1 has no heating function and can not simulate the sidestream smoke that needs to be heated. However, the present invention can well simulate the using experience of traditional flue-cured tobacco, both the mouth inhalation material and nasal inhalation material of the e-cigarettes can be heated and then be inhaled by the mouth and nose respectively.

Only the flavor (16) that volatilizes naturally can be used in WO2011109848A1. However, due to the heating function and the blowing function of the nasal inhalation module of the present invention, the nasal inhalation material can be easily extended to essence, nicotine or other alkaloids, nicotine salt, e-liquid, tobacco extract paste, tobacco extract liquid, tobacco smoke condensate, cannabinoids, solvents, and more.

The flavor reservoir (6) was implanted completely in the inhaler component and the location of the flavor reservoir (6) must be very close to the user's nose in WO2011109848A1. However, the nasal inhalation module in the present invention can be arranged on the shell of the mouth inhalation module, as well as being partially implanted inside of the mouth inhalation module because of the volatiles conduit, which can further precisely send the volatiles to the user's nasal cavity and make the location of chamber for the mouth inhalation module more flexible in the e-cigarettes.

The beneficial effects of the present invention are as follows: an e-cigarette used by cooperation of mouth and nose is disclosed, wherein the essence and nicotine of the e-liquid, HNB solid cartridge or paste material can be partially or completely transferred from the mouth inhalation module into the nasal inhalation module of the e-cigarette. While the mouth normally inhaling the vapor aerosol or HNB smoke, and at the same time, the essence and nicotine in the nasal inhalation module can be directly inhaled into the nasal cavity by the nose, without being diluted and intercepted by many intermediate organs of “small circulation through mouth” or “large circulation through lungs” with the vapor aerosol or HNB smoke that inhaled by mouth, therefore, it can improve the identification accuracy of essences by the olfactory nerve in the nasal cavity, whereby improving the flavor reproduction level of e-cigarettes, as well as improving the transmission efficiency and utilization rate of nicotine, reducing the nicotine intake each time for nicotine satisfaction, and reducing the risk of the inhalation of essence, essence solvents and related substances into lungs caused by using e-cigarettes. Moreover, the variety of nasal inhalation materials can be increased for the heating function and blowing function in the nasal inhalation module of the e-cigarettes, as well as the improvement of olfactory perception of the essence, nicotine and other nasal inhalation materials especially in low temperature environments, whereby the using experience of e-cigarettes of the present invention is very close to that of traditional flue-cured tobacco.

### BRIEF DESCRIPTION OF THE DRAWINGS

In order to more clearly illustrate the technical solutions in the embodiments of the present invention, the accompanying drawings that need to be used in the description of the embodiments will be briefly described below, and it is obvious that the accompanying drawings in the following description are only some of the embodiments of the present invention, and other drawings can be obtained by those skilled in the art based on these accompanying drawings without creative work, wherein:

FIG. 1 is a structural frame diagram of an e-cigarette containing a mouth inhalation module and a nasal inhalation module, and the components are as follows: 11 inhalation nozzle module, 12 atomizing or heating module, 13 circuit module, 14 display module, 15 nasal inhalation module.

FIG. 2 is the frame diagram of the component structure of nasal inhalation module 15, 1501 nasal inhalation material carrier, 1502 nasal inhalation material carrier cover, 1503 nasal inhalation material volatiles conduit, 1504 conduit extension tube, 1505 fan, 1506 fan controller, 1507 electric heater, 1508 electric heater controller, 1509 fan circuit, 1510 electric heater circuit, 1511 battery terminal wire interface, 1512 battery, 1513 component retainer.

FIG. 3 is an external view of the e-cigarette in the Embodiment 1.

FIG. 4 is an exploded view of the structure of the e-cigarette in the Embodiment 1, and the components are as follows: 100 inhalation nozzle, 101 e-cigarette set, 102 first nasal inhalation material bottle, 103 first nasal inhalation material bottle cap, 104 second nasal inhalation material bottle, 105 second nasal inhalation material bottle cap, 106 double-sided adhesive tape, 107 the first battery, 108 the first battery terminal wire interface, 109 atomizer circuit.

FIG. 5 is the external view of the e-cigarette in the Embodiment 2.

FIG. 6 is an exploded view of the structure of the e-cigarette in the Embodiment 2, and the components are as follows: 200 atomizing cartridge, 201 cigarette rod, 202 nasal inhalation material bottle, 203 nasal inhalation material bottle cap, 204 chamber cover, 205 nasal inhalation material bottle cap alignment cover hole, 206 chamber cover slot, 207 fan, 208 fan knob switch, 209 fan knob switch alignment cover hole, 210 the first battery, 211 the first battery terminal wire interface, 212 wires, 213 atomizer circuit, 214 chamber.

FIG. 7 is an external view of the e-cigarette in the Embodiment 3.

FIG. 8 is an exploded view of the structure of the e-cigarette in the Embodiment 3, and the components are as follows: 300 atomizing cartridge, 301 cigarette rod, 302 the first battery, 303 the first battery terminal wire interface, 304 atomizer circuit, 305 the second battery, 306 the second battery terminal wire interface, 307 wires, 308 fan, 309 fan controller, 310 mesh electric heating plate, 311 electric heating plate controller, 312 nasal inhalation material, 313 chamber cover, 314 electric heating plate controller chamber cover hole, 315 fan controller cover hole, 316 the second battery charging port, 317 chamber, 318 nasal inhalation material volatiles outlet.

FIG. 9 is an external view of the e-cigarette in the Embodiment 4.

FIG. 10 is an exploded view of the structure of the e-cigarette in the Embodiment 4, and the components are as follows: 400 atomizing cartridge, 401 cigarette rod, 402 the first battery, 403 the first battery terminal wire interface, 404 atomizer circuit, 405 chamber, 406 the second battery, 407 the second battery terminal wire interface, 408 wires, 409 electric heating pot, 410 electric heating pot knob switch, 411 electric heating pot sealing cover, 412 chamber cover, 413 electric heating pot knob switch cover hole, 414 nasal inhalation material volatiles conduit interface, 415 nasal inhalation material volatiles conduit, 416 conduit extension tube.

FIG. 11 is an external view of the e-cigarette in the Embodiment 5.

FIG. 12 is an exploded view of the structure of the e-cigarette in the Embodiment 5, and the components are as follows: 500 atomizing cartridge, 501 cigarette rod, 502 the first battery, 503 the first battery terminal wire interface, 504 atomizer circuit, 505 base, 506 magnetic column in the base, 507 thimble electrode in the base, 508 the first battery charging port, 509 electric heating plate, 510 nasal inhalation material pot, 511 nasal inhalation material pot fixing ring, 512 nasal inhalation material pot spout plug, 513 shell hole for the alignment of nasal inhalation material pot spout plug, 514 electric heating plate knob switch, 515 shell hole for alignment of electric heating plate knob switch, 516 nasal inhalation material bottle, 517 nasal inhalation material bottle cap, 518 shell hole for the alignment of nasal inhalation material bottle cap, 519 fan, 520 fan knob switch, 521 shell hole for the alignment of fan knob switch, 522 nasal inhalation material volatiles conduit, 523 nasal inhalation material volatiles conduit assembly interface, 524 conduit extension tube, 525 wires for electric heating plate and fan, 526 magnetic column in the atomizing cartridge, 527 electrode tip in the atomizing cartridge.

FIG. 13 is an external view of the e-cigarette in the Embodiment 6.

FIG. 14 is an exploded view of the structure of the e-cigarette in the Embodiment 6, and the components are as follows: 600 filter tip, 601 solid cartridge, 602 cartridge sleeve, 603 heating element base, 604 heating element, 605 heating element electrical connection port, 606 the first battery, 607 the first battery terminal wire interface, 608 heating element circuit, 609 the first battery charging port, 610 double-sided adhesive tape, 611 the second battery, 612 the second battery terminal wire interface, 613 wires, 614 fan knob switch, 615 fan, 616 electric heating plate, 617 electric heating plate knob switch, 618 the first nasal inhalation material bottle, 619 the first nasal inhalation material bottle cap, 620 heating element switch, 621 the second nasal inhalation material bottle, 622 the second nasal inhalation material bottle cap, 623 cigarette rod.

FIG. 15 is an external view of the e-cigarette in the Embodiment 7.

FIG. 16 is an exploded view of the structure of the e-cigarette in the Embodiment 7, and the components are as follows: 700 filter tip, 701 cartridge filled with paste material, 702 cartridge sleeve, 703 heating element base, 704 heating element, 705 heating element electrical connection port, 706 the first battery, 707 the first battery charging port, 708 the first battery terminal wire interface, 709 heating element circuit, 710 heating element switch, 711 electric heating plate, 712 fan, 713 electric heating plate knob switch, 714 fan knob switch, 715 wires, 716 nasal inhalation material pot fixing ring, 717 nasal inhalation material pot, 718 nasal inhalation material pot airtight cover with volatiles outlet valve, 719 chamber cover, 720 push-type switch, 721 nasal inhalation material volatiles conduit, 722 conduit extension tube, 723 shell hole on the chamber cover for the alignment of electric heating plate knob switch, 724 shell hole on the chamber cover for the alignment of fan knob switch, 725 chamber, 726 cigarette rod.

Remarks, wires are used in the above figures to represent the atomizer circuit and heating element circuit in the atomizing or heating module 12, and these circuits are usually connected with conventional components or electronics used in mouth inhalation modules such as PCBA, starter and sensor.

### MODE OF CARRYING OUT THE INVENTION

The following will clearly describe the technical solutions in the embodiments of the present invention. Obviously, the described embodiments are only some of the embodiments of the present invention, rather than all the embodiments. Based on the embodiments of the present invention, all other embodiments obtained by person of ordinary skill in the art without making creative effort belong to the protection scope of the present invention.

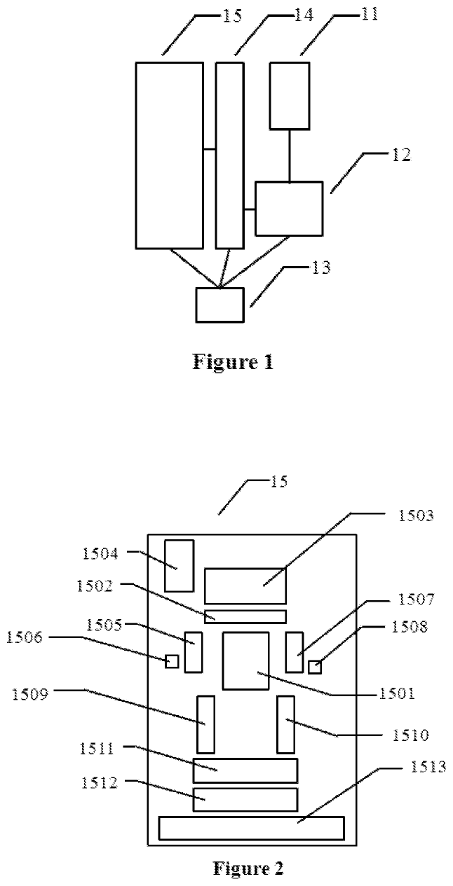

Referring to FIG. 1-FIG. 16 of the accompanying drawings, the embodiments of the present invention include:

### Comparative Example 1

Prepare an ordinary e-liquid and mark it as e-liquid A, and the mass proportion of each component is as follows: 3% benzoate nicotine salt, 2% water, 43% propylene glycol, 39% glycerol, and 2% sucralose sweetener, 1% WS-23 cooling agent essence and 10% passion fruit essence, and 2 g e-liquid A is injected into an empty atomizing cartridge, forming a set of conventional vapor e-cigarette with a matching cigarette rod, after the atomizing cartridge being inserted into the chamber and connected with the e-cigarette rod, the aerosol generated from the atomization of the e-liquid A can be normally inhaled by mouth.

### Comparative Example 2

A HNB e-cigarette containing tobacco essence and nicotine in the solid cartridge is purchased from the market, after connecting the solid cartridge, the cartridge sleeve, and the cigarette rod with a sheet-shaped heating element and its base, pushing the circuit switch of the heating element, the smoke generated from the solid cartridge being heated up can be normally inhaled by mouth.

Embodiment 1: Disposable e-cigarette used by cooperation of mouth and nose, and e-liquid A and the partial of essences are transferred into the nasal inhalation module.

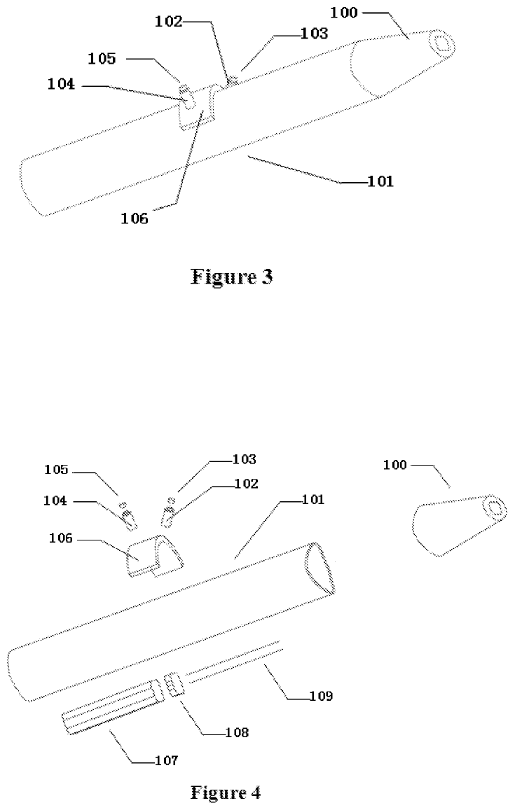

E-liquid B is prepared by deleting the mass proportion of passion fruit essence in e-liquid A. The mass proportions of the components in e-liquid B are as follows: 3% benzoate nicotine salt, 2% water, 43% propylene glycol, 49% glycerol, 2% sucralose sweetener, 1% WS-23 cooling agent essence. 2 g e-liquid B is injected into a disposable vapor e-cigarette set 101, wherein 0.2 g passion fruit essence has been transferred out comparing to 2 g e-liquid A. The mouth inhalation module, which is comprised of the first battery 107, the first battery terminal wire interface 108, atomizer circuit 109, inhalation nozzle 100 and other components, enables the e-liquid B to be atomized normally, and the user can inhale the atomized aerosol by mouth. As shown in FIG. 3 and FIG. 4 of the accompanying drawings, two small nasal inhalation material bottles are fixed on the e-cigarette set by a layer of double-sided adhesive tape 106 pre-adhered on the surface of the e-cigarette rod, and 0.2 g passion fruit essence are dropped into the first nasal inhalation material bottle 102, and 10 drops of e-liquid A are dropped into the second nasal inhalation material bottle 104, then two small stainless steel balls that have been preheated to 80° C. are placed into these two nasal inhalation material bottles respectively as the heater of the heating compound with non-electrical energy and without using battery, whereby making a simple nasal inhalation module 15 constituted with the double-sided adhesive tape, two nasal inhalation material bottles, two small hot balls, the contained e-liquid A and 0.2 g passion fruit essence. When using the e-cigarette, the user inhales the atomized aerosol generated from the e-liquid B by mouth, at the same time, choosing one or opening the first nasal inhalation material bottle cap 103 and the second nasal inhalation material bottle cap 105 together, and inhales the volatiles of passion fruit essence and e-liquid A volatilized from the two nasal inhalation material bottles, so as to use the e-cigarette by cooperation of mouth and nose.

Embodiment 2: A cartridge-changing e-cigarette used by cooperation of mouth and nose, and the essences and nicotine are partially transferred into the nasal inhalation module.

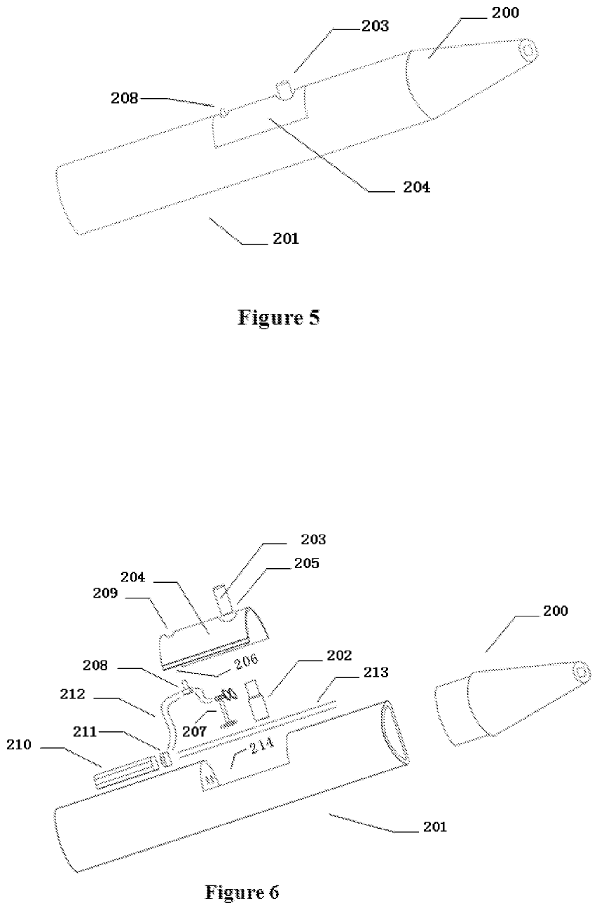

E-liquid C is prepared by removing the cooling agent essence, passion fruit essence and 1% benzoate nicotine salt from e-liquid A but retaining the sweetener essence. The mass proportions of the components in e-liquid C are as follows: 2% benzoate nicotine salt, 3% water, 43% propylene glycol, 50% glycerol, 2% sucralose sweetener, and 2 g of the e-liquid C is injected into an empty atomizing cartridge, wherein 0.2 g passion fruit essence, 0.02 g of WS-23 cooling agent essence and about 0.011 g of nicotine are transferred out comparing to 2 g e-liquid A. The cigarette rod 201 of the cartridge-changing e-cigarette that matched with the interface of the atomizing cartridge is lengthened by 30 mm, after fine-tuning the position of the original circuit module and atomizing or heating module, including sensor, PCBA, wires, and the likes, inside of the cigarette rod, an chamber 214 with a length of 30 mm, a width of 15 mm, and a depth of 4 mm in the center is made in the middle of the cigarette rod, and the chamber can be opened or closed by a chamber cover 204 and a chamber cover slot 206 with corresponding size. The mouth inhalation module, which is comprised of the first battery 210, the first battery terminal wire interface 211, the atomizer circuit 213, the atomizing cartridge 200 and other components, enables the e-liquid C to be atomized normally, and the user can inhale the atomized aerosol by mouth. As shown in FIG. 5 and FIG. 6 of the accompanying drawings, a miniature nasal inhalation material bottle 202 containing a mixture of 0.2 g passion fruit essence, 0.02 g of WS-23 cooling agent essence and 0.011 g of nicotine is implanted and fixed into the chamber, as well as a fan 207 aligned with the spout of nasal inhalation material bottle and the user's face; A U shape two-layer cloth bottle sleeve filled with calcium oxide particles wrapping around the side and bottom of the nasal inhalation material bottle as a heater of the heating component, whereby a chemical exothermic reaction with water in air or directly added by the user can heat the nasal inhalation material with non-electrical energy and without using battery. An elongated nasal inhalation material bottle cap 203 is arranged on the nasal inhalation material bottle 202 and extends from the chamber 214 to the outside of the chamber cover 204, a fan knob switch 208 for controlling the opening and rotating speed of the fan is arranged between the chamber and the chamber cover, and an nasal inhalation material bottle cap alignment cover hole 205 and a fan knob switch alignment cover hole 209 are provided on the chamber cover for convenient operation by the user from the outside of the chamber cover; A set of wires 212 are led from the output interface of the first battery to connect with the fan knob switch and the fan; the nasal inhalation material bottle, fan, knob switch and other components then constitute the nasal inhalation module 15, sharing the first battery with the mouth inhalation module. When using the e-cigarette, the user inhales the atomized aerosol generated from the e-liquid C by mouth, and at the same time, the user may unscrew the cap of the nasal inhalation material bottle and turn on the fan from the outside of the chamber cover, and inhales the volatiles of the WS-23 cooling agent essence, passion fruit essence and nicotine by nose, which are blown out from the nasal inhalation material bottle by the fan and the heater, so as to use the e-cigarette by cooperation of mouth and nose.

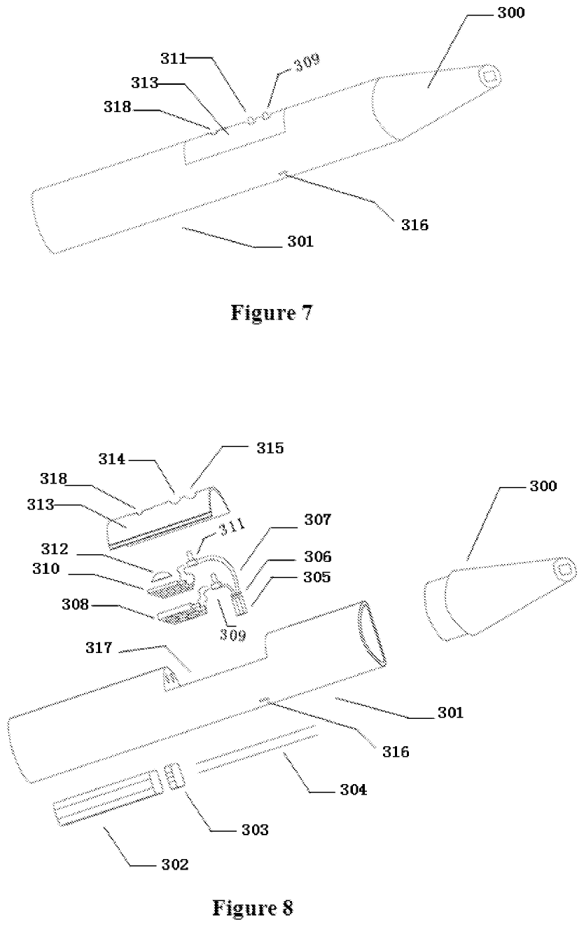

Embodiment 3: A closed type cartridge-changing e-cigarette used by cooperation of mouth and nose, the essences are completely transferred into the nasal inhalation module, and the essence solvents and nicotine salt are partially transferred into the nasal inhalation module. E-liquid D is prepared by removing the cooling agent essence, passion fruit essence, sweetener essence and partial nicotine salt from e-liquid A. The mass proportions of the components in e-liquid D are as follows: 2% benzoate nicotine salt, 6% water, 40% propylene glycol, and 52% glycerol, and 2 g of the e-liquid D is injected into an empty atomizing cartridge, wherein 0.2 g passion fruit essence, 0.02 g benzoate nicotine salt, 0.06 g propylene glycol, 0.02 g WS-23 cooling agent essence, and 0.04 g sweetener are transferred out comparing to 2 g e-liquid A. The cigarette rod 301 of the cartridge-changing vapor e-cigarette that matched with the interface of the atomizing cartridge 300 is lengthened by 40 mm, after fine-tuning the position of the original circuit module and atomizing or heating module, including sensor, PCBA, wires, and the likes, inside of the cigarette rod, an chamber 317 with a length of 40 mm, a width of 15 mm, and a depth of 6 mm in the center is made in the middle of the cigarette rod 301, and the chamber can be opened or closed by a chamber cover 313 and a chamber cover slot with corresponding size. The mouth inhalation module, which is composed of the first battery 302, the first battery terminal wire interface 303, the atomizer circuit 304, the atomizing cartridge 300 and other components, enables the e-liquid D to be atomized normally, and the user can inhale the atomized aerosol by mouth. As shown in FIG. 7 and FIG. 8 of the accompanying drawings, a new lithium-ion battery, namely the second battery 305, is implanted and fixed into the chamber perpendicular to the direction of the cigarette rod, and a charging port 316 is provided for the second battery at the corresponding position of the cigarette rod, two sets of wires 307 are led out through the second battery terminal wire interface 306 to connect a disk-type fan 308 and a mesh electric heating plate 310 in the chamber respectively, a fan controller 309 with knob switch for controlling the fan opening and rotating speed is set between the chamber and the fan controller cover hole 315, and an electric heating plate controller 311 with knob switch for controlling the opening and the temperature level of the mesh electric heating plate is set between the chamber and the corresponding chamber cover hole 314, which is convenient for users to operate from the outside of the chamber cover. Some fresh orange peel, 0.2 g passion fruit essence, 0.02 g benzoate nicotine salt, 0.06 g propylene glycol, 0.02 g WS-23 cooling agent essence and 0.04 g sweetener are mixed as nasal inhalation material 312 and placed on the mesh electric heating plate, and the aroma of orange peel can be released from the nasal inhalation material volatiles outlet 318 preset at the corresponding position of the chamber cover. The second battery, the fan, the electric heating plate, the nasal inhalation material, the knob switch and other components then constitute the nasal inhalation module 15. When using the e-cigarette, the user inhales the atomized aerosol generated from the e-liquid D by mouth, and at the same time, the electric heating plate and fan can be controlled from the outside of the chamber cover, and the volatiles of the nasal inhalation material on the electric heating plate blown out by the fan can be inhaled by nose, so as to use the e-cigarette by cooperation of mouth and nose.

Embodiment 4: A open type cartridge-changing e-cigarette used by cooperation of mouth and nose, the essences and nicotine salt are completely transferred into the nasal inhalation module, essence solvents are partially transferred into the nasal inhalation module and further adding new substances as nasal inhalation material.

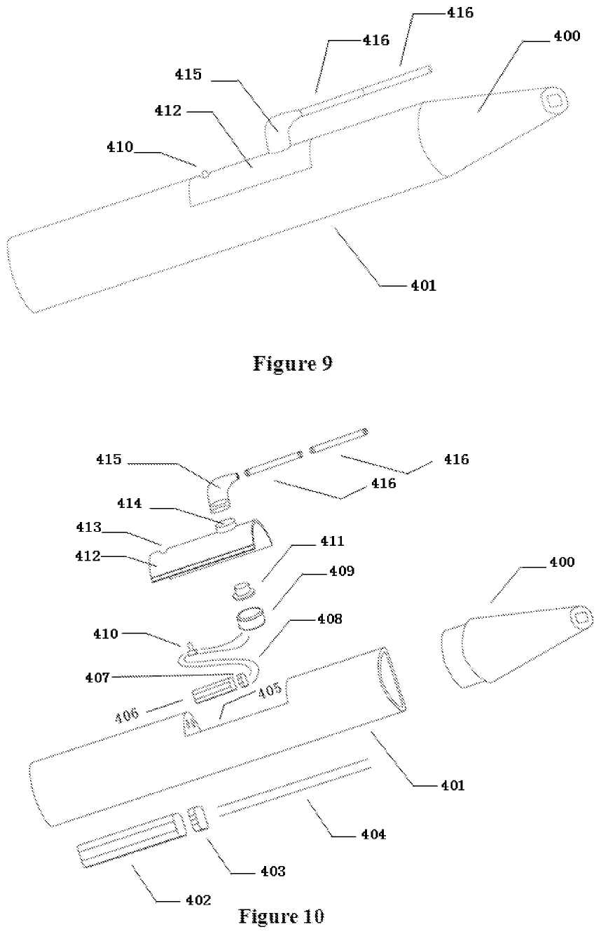

E-liquid E is prepared by removing the cooling agent essence, passion fruit essence, sweetener essence and essence solvent propylene glycol from e-liquid A. The mass proportions of the components in e-liquid E are as follows: 6% water, 40% propylene glycol, and 54% glycerol. 2 g e-liquid E is injected into an empty atomizing cartridge with self-service injection port, wherein 0.2 g passion fruit essence, 0.06 g benzoate nicotine salt, 0.06 g propylene glycol, 0.02 g WS-23 cooling agent essence, and 0.04 g sweetener are transferred out comparing to 2 g e-liquid A. The cigarette rod 401 of the cartridge-changing vapor e-cigarette that matched with the interface of the atomizing cartridge 400 is lengthened by 10 mm, after fine-tuning the position of the original circuit module and atomizing or heating module, including sensor, PCBA, wires, and the likes, inside of the cigarette rod, an chamber 405 with a length of 10 mm, a width of 10 mm, and a depth of 5 mm in the center is made in the middle of the cigarette rod, and the chamber can be opened or closed by a chamber cover 412 and a chamber cover slot with corresponding size. The mouth inhalation module, which is composed of the first battery 402, the first battery terminal wire interface 403, the atomizer circuit 404, the atomizing cartridge 400 and other components, enables the e-liquid D to be atomized normally, and the user can inhale the atomized aerosol by mouth. As shown in FIG. 9 and FIG. 10 of the accompanying drawings, a non-rechargeable button battery, acting as the second battery 406, is implanted and fixed into the chamber, and a set of wires 408 are led out through the second battery terminal wire interface 407 to connect a miniature electric heating pot 409 and an electric heating pot knob switch 410, and the electric heating pot is equipped with a resistance heating component. Some ethyl maltol essence powder and cooked pasty mutton fat are mixed first according to the mass ratio of 3:7, and then mix with 0.2 g passion fruit essence, 0.06 g benzoate nicotine salt, 0.06 g propylene glycol, 0.02 g WS-23 cooling agent essence but excepting the transferred sweetener, and further mix with some tobacco smoke condensate and 0.01 g of cannabidiol to obtain a final mixture as the nasal inhalation material and put into the electric heating pot. The knob switch 410 for controlling the opening and the temperature level of the electric heating pot is set between the chamber cover 412 and the chamber 405, and the knob switch can be operated from the electric heating pot knob switch cover hole 413. A nasal inhalation material volatiles conduit interface 414, a nasal inhalation material volatiles conduit 415 and a conduit extension tube 416 are set in the chamber cover corresponding to the position of the spout of the electric heating pot, which are connected to each other by a thread at the end. The second battery, electric heating pot, the knob switch, nasal inhalation material, volatiles conduit assembly and other components then constitute the nasal inhalation module 15. When using the e-cigarette, the user inhales the atomized aerosol generated from the e-liquid E by mouth, and at the same time, the user can firstly open the chamber cover to take out the electric heating pot sealing cover 411, then close the chamber cover, and then control the knob switch of the heating pot from the outside of the chamber cover, and inhales the nasal inhalation material volatiles, released from the heating pot and led to nose by the nasal inhalation material volatiles conduit assembly, so as to use the e-cigarette by cooperation of mouth and nose.

Embodiment 5: A closed type cartridge-changing e-cigarette used by cooperation of mouth and nose, the essences are completely transferred into the nasal inhalation module, and the essence solvents are partially transferred into the nasal inhalation module, and further adding new substances as nasal inhalation material.

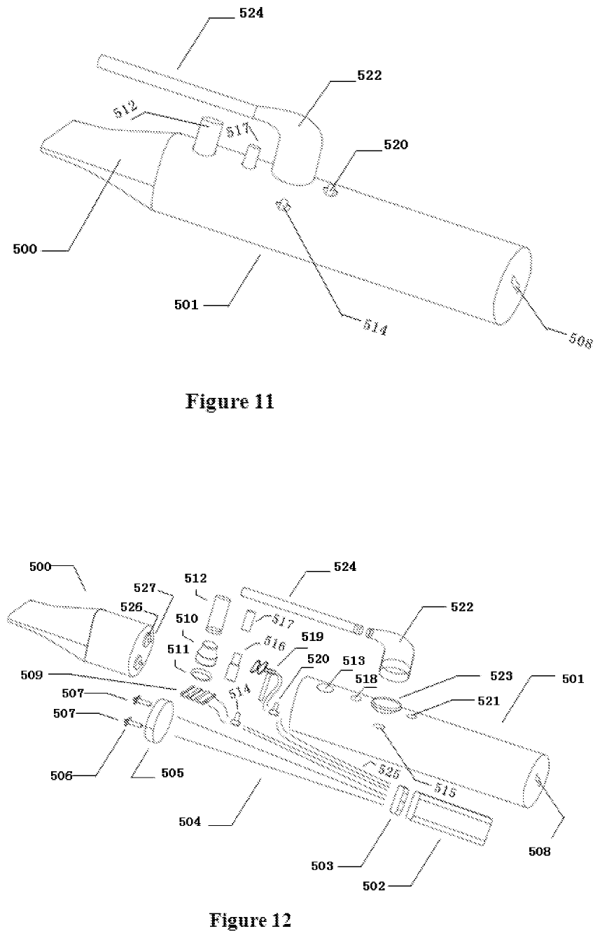

E-liquid F is prepared by removing the cooling agent essence, passion fruit essence, sweetener essence, essence solvents propylene glycol and glycerol from e-liquid A. The mass proportions of the components in e-liquid F are 3% benzoate nicotine salt and 97% water, and 2 g e-liquid F is injected into an empty atomizing cartridge without self-service injection port, wherein 0.2 g passion fruit essence, 0.86 g propylene glycol, 0.78 g glycerol, 0.02 g WS-23 cooling agent essence, and 0.04 g sweetener are transferred out comparing to 2 g e-liquid A. The shell of original cigarette rod is extended by 100 mm to the side of the atomizing cartridge to form a long chamber, and the components of the atomizer circuit 504, the base 505 containing a pair of magnetic columns 506 and a pair of thimble electrodes 507 are moved forward 100 mm in the direction of the atomizing cartridge and correspond to the magnetic column 526 and the electrode tip 527 in the atomizing cartridge respectively. The mouth inhalation module, which is composed of the atomizing cartridge 500, the extended cigarette rod 501, the sensor and PCBA in the original circuit module, the first battery 502, the first battery terminal wire interface 503, the extension wire and other components, enables the e-liquid F to be atomized normally, and the user can inhale the atomized aerosol by mouth. As shown in FIG. 11 and FIG. 12 of the accompanying drawings, two sets of wires 525 are led from the first battery terminal wire interface 503 to connect the electric heating plate 509, the electric heating plate knob switch 514, the fan 519 and the fan knob switch 520 respectively, which are set in the chamber, the extended cigarette rod 501 has shell hole 515 and another shell hole 521 corresponding to the positions of the two knob switches respectively, so that the user can control the electric heating plate and the fan from the outside of the cigarette rod. A nasal inhalation material pot 510, a nasal inhalation material pot fixing ring 511, and an elongated nasal inhalation material pot spout plug 512 are sequentially provided on the electric heating plate 509, wherein the nasal inhalation material pot fixing ring is fixed on the electric heating plate by screws, and the nasal inhalation material pot spout plug 512 is extended outside the e-cigarette rod through the alignment shell hole 513, 0.2 g passion fruit essence, 0.5 g propylene glycol, 0.02 g WS-23 cooling agent essence, but excepting the transferred sweetener and glycerol, are added to the nasal inhalation material pot. A nasal inhalation material bottle 516 with a threaded spout is re-implanted and fixed into the chamber, and an elongated nasal inhalation material bottle cap 517 with a corresponding thread is extended outside the cigarette rod through the alignment hole 518 of the cigarette rod shell. Users can open or close the nasal inhalation material pot spout plug and the nasal inhalation material bottle cap through the outside of the e-cigarette rod, and an appropriate amount of arecoline and tobacco extract paste are added into the nasal inhalation material bottle. An opening is provided for the chamber as the nasal inhalation material volatiles conduit assembly interface 523 on the shell of the cigarette rod, and a nasal inhalation material volatiles conduit 522 and a conduit extension tube 524 are arranged, which are connected to each other by threads preset at the ends. The electric heating plate, nasal inhalation material, nasal inhalation material pot, nasal inhalation material pot spout plug, nasal inhalation material bottle, nasal inhalation material bottle cap, fan, knob switch, wire, nasal inhalation material volatiles conduit assembly, and other components then constitute the nasal inhalation module 15. When using the e-cigarette, the user inhales the atomized aerosol generated from the e-liquid F by mouth, and at the same time, the user can open the nasal inhalation material pot spout plug and the nasal inhalation material bottle cap, so as to release the volatiles of passion fruit essence, WS-23 cooling agent essence, arecoline and tobacco extract paste from the three outlets, including the shell hole 513 aligned with the nasal inhalation material pot spout plug, the shell hole 518 aligned with the nasal inhalation material bottle cap, as well as the nasal inhalation material volatiles conduit assembly composed of nasal inhalation material volatiles conduit 522, nasal inhalation material volatiles conduit assembly interface 523, conduit extension tube 524. The two outlets of nasal inhalation material volatiles that the shell hole 513 for the alignment of nasal inhalation material pot spout plug and the shell hole 518 for the alignment of nasal inhalation material bottle cap may be covered with fingers, so that the mixed nasal inhalation material volatiles can be released from the outlet of the conduit assembly individually, and the speed and temperature at which the nasal inhalation material volatiles from the chamber entering the nasal cavity of the user can be adjusted through outside of the cigarette rod by turning on the electric heating plate knob switch 514 and the fan knob switch 520, so as to use the e-cigarette by cooperation of mouth and nose.

Embodiment 6: HNB e-cigarette used by cooperation of mouth and nose, and the essences and nicotine are partially transferred into the nasal inhalation module.

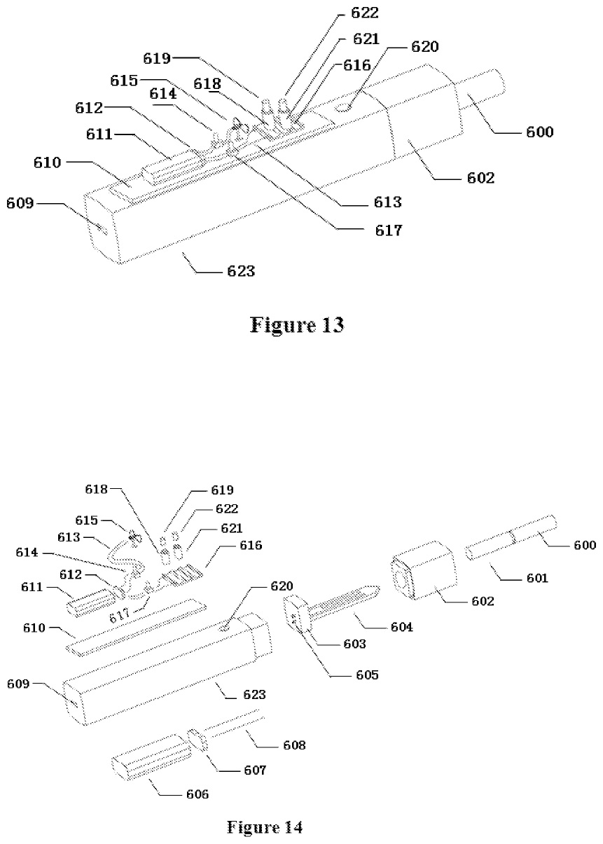

An ordinary HNB e-cigarette that using solid cartridge is purchased from the market, wherein components of the mouth inhalation module mainly include cigarette rod 623, filter tip 600, solid cartridge 601, cartridge sleeve 602, heating element base 603, heating element 604, heating element electrical connection port 605, the first battery 606, the first battery terminal wire interface 607, heating element circuit 608, the first battery charging port 609, heating element switch 620, and the likes, and using a solid cartridge containing only a small amount of essence substances and nicotine, for example a solid cartridge filled of sheets with a small amount of plant-extracted nicotine and tobacco essence, and the smoke generated from the solid cartridge after being heated by the heating element can be normally inhaled by mouth. As shown in FIG. 13 and FIG. 14 of the accompanying drawings, a piece of double-sided adhesive tape 610 with a width similar to that of the e-cigarette is adhered on the surface of the cigarette rod 623 slightly lower than the heating element switch 620, and components such as the second battery 611, the second battery terminal wire interface 612, fan 615, fan knob switch 614, electric heating plate 616 and electric heating plate knob switch 617 are sequentially adhered and fixed on surface of the double-sided adhesive tape, and two sets of wires 613 are led out from the second battery terminal wire interface 612 to connect with the fan, the fan knob switch, the electric heating plate and the electric heating plate knob switch respectively, and two nasal inhalation material bottles are fixed by screws on the electric heating plate, the first nasal inhalation material bottle 618 is provided with an appropriate amount of tobacco essence, and the second nasal inhalation material bottle 621 is provided with some tobacco extract liquid. The second battery, electric heating plate, fan, nasal inhalation material bottle, knob switch and double-sided adhesive tape then constitute the nasal inhalation module 15, which is exposed on the HNB e-cigarette rod. When using the e-cigarette, the user inhales the smoke generated from the HNB mouth inhalation module, and at the same time, the user may open the first nasal inhalation material bottle cap 619 and the second nasal inhalation material bottle cap 622, so that the volatiles of the tobacco essence and the tobacco extract liquid enter into the user's nasal cavity directly from the two nasal inhalation material bottles, and the user may further turn on and control the fan knob switch as well as the electric heating plate knob switch to adjust the speed and temperature of the nasal inhalation material volatiles from the nasal inhalation module entering into the nasal cavity, so as to use the e-cigarette by cooperation of mouth and nose.

Embodiment 7: HNB e-cigarette used by cooperation of mouth and nose, and the essences and nicotine are completely transferred into the nasal inhalation module.

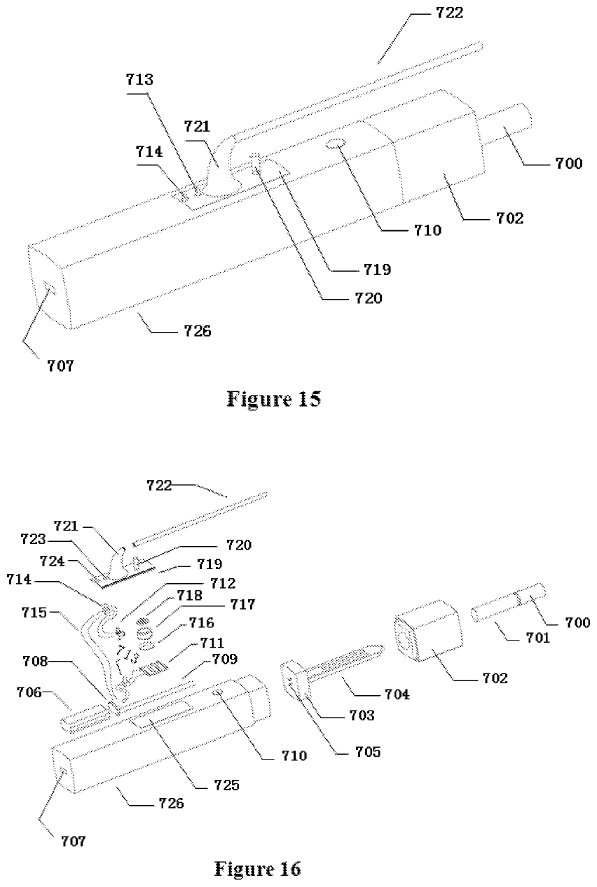

Extending the shell of the Comparative Example 2 or similar HNB e-cigarette set by 50 mm, and fine-tuning the positions of the original circuit module and atomizing or heating module, including PCBA, wires, temperature sensor, and the likes, inside of the HNB e-cigarette rod, an chamber 725 with a length of 50 mm, a width of 12 mm, and a depth of 10 mm in the center is made in the middle of the cigarette rod 726, and the chamber can be opened or closed by a chamber cover 719 and a chamber cover slot with corresponding size. The components of the HNB mouth inhalation module mainly include filter tip 700, cartridge filled with paste material 701, cartridge sleeve 702, heating element base 703, heating element 704, heating element electrical connection port 705, the first battery 706, the first battery charging port 707, the first battery terminal wire interface 708, the heating element circuit 709, the heating element switch 710, and the likes, and using paste material without essence substances and nicotine, for example, paste material that made from plant herbs, users can normally inhale the smoke by mouth which is generated from the paste material after being heated by the heating element. As shown in FIG. 15 and FIG. 16 of the accompanying drawings, a fan 712, an electric heating plate 711, an nasal inhalation material pot fixing ring 716, an nasal inhalation material pot 717 and an nasal inhalation material pot airtight cover with volatiles outlet valve 718 are sequentially implanted and fixed into the chamber, and two sets of wires led from the first battery terminal wire interface 708 connect with the fan 712, the fan knob switch 714, the electric heating plate 711, and the electric heating plate knob switch 713 respectively, and the user operation portions of the two knob switches are fixed on the chamber cover 719 through the shell hole 723 for the alignment of electric heating plate knob switch and the shell hole 724 for the alignment of fan knob switch, which are convenient for the user to control the electric heating plate and the fan from outside the chamber. A push-type switch 720 for connecting the volatiles outlet valve of the airtight cover of the nasal inhalation material pot is arranged on the chamber cover, and some tobacco smoke condensate with opaque paste appearance and strong tar odor, is added to the nasal inhalation material pot, closing the nasal inhalation material pot airtight cover 718, and the volatiles outlet valve of the nasal inhalation material pot airtight cover can be opened or closed by the push-type switch 720 on the chamber cover 719. The push-type switch 720 and the nasal inhalation material pot airtight cover with volatiles outlet valve, can be further simplified as a carrying rope and an exhaust valve similar to that on an electric cooker airtight cover or on a pressure cooker airtight cover, and the volatiles outlet valve can be opened or closed with the control of the carrying rope. An nasal inhalation material volatiles conduit 721 is further provided on the chamber cover 719, which is connected with an conduit extension tube 722 by an end thread. The electric heating plate, the nasal inhalation material pot, the nasal inhalation material pot airtight cover with volatiles outlet valve, the push-type switch of the nasal inhalation material pot airtight cover, the fan, the knob switch, and the conduit assembly then constitute the nasal inhalation module 15, which shares the first battery with the mouth inhalation module. When using the e-cigarette, the user inhales the smoke generated from the HNB mouth inhalation module by mouth, and at the same time, the push-type switch 720 over the chamber cover can be used to open the volatiles outlet valve of the airtight cover of the nasal inhalation material pot, so that the nasal inhalation material volatiles in the nasal inhalation material pot can be led out to the user's nasal cavity by the nasal inhalation material volatiles conduit 721 and the conduit extension tube 722.

The invention has the following effects:

The nasal inhalation module is fixed on the shell of mouth inhalation module of the e-cigarette by using the double-sided adhesive tape, screws, and the likes in the Embodiment 1 and the Embodiment 6 respectively, and the nasal inhalation module is partially or completely implanted into a chamber in the middle of the mouth inhalation module by appropriately lengthening the shell of e-cigarette set in the Embodiment 2, 3, 4, and 7 respectively, and the nasal inhalation module is partially implanted into a properly lengthened chamber of the e-cigarette rod connecting to the atomizing cartridge and the base, magnetic column, electrode, wires are shifted forward accordingly in the Embodiment 5.

The second battery is employed for the electrical appliances of the nasal inhalation module in the Embodiment 3, 4, and 6, while the first battery in the e-cigarette set is shared by the nasal inhalation module with the mouth inhalation module in the Embodiment 2, 5, and 7, and there is no battery for the nasal inhalation module in the Embodiment 1.

The nasal inhalation material can be heated by heating component with electrical energy in the Embodiment 3, 4, 5, 6 and 7. Two small hot balls act as the heater of the heating component in the Embodiment 1, and a cloth bottle sleeve filled with chemical exothermic reactant of calcium oxide particles act as the heater of the heating component in the Embodiment 2, both of which can heat the nasal inhalation material with non-electrical energy and without using battery.

One end of the volatiles conduit is close to the nasal inhalation material carrier in the Embodiment 4, and indirectly connected with the nasal inhalation material carrier in the

Embodiment 5, and directly connected with the nasal inhalation material carrier in the Embodiment 7.

From the Embodiments 1-5, the flavor essence, the sweetener essence, the cooling agent essence, the essence solvent propylene glycol, and the essence solvent glycerol are transferred out sequentially from the e-liquid A of the Comparative Example 1. The e-liquids used in the Embodiment 2, 3 and 4 further reduce the nicotine or nicotine salt from the e-liquid A, and the HNB cartridge in the Embodiment 6 and 7 contain less essence and nicotine than those in the HNB cartridge of the Comparative Example 2. From the Embodiments 1-7, the nicotine, nicotine salt, essence, essence solvent are partially or completely transferred from the mouth inhalation module into the nasal inhalation module of e-cigarette in the present invention, so the essence, essence solvent and related substances in the atomized aerosol and HNB smoke entering the lungs from mouth are greatly decreased or even zero, whereby helping users to reduce the risk of essences, essence solvents and essence-related substances entering to the lungs and to improve the using safety of e-cigarette. In addition, new nasal inhalation materials such as fresh orange peel, ethyl maltol essence and mutton essence, cannabinol, arecoline, tobacco extract paste, tobacco extract liquid and tobacco smoke condensate are added to the nasal inhalation module in some embodiments, which improves the using experience of e-cigarettes.

In addition, by providing a nasal inhalation module in the e-cigarette, and the essences are partially transferred or completely transferred from the mouth inhalation module into the nasal inhalation module of e-cigarettes, user's nasal cavity can directly inhale the essence aroma and quickly transfer signal to the brain, whereby improving the accurate recognition of essence molecules and the flavor reproduction level of e-cigarettes. Moreover, these essences do not need to undergo the process of being heated at high temperature together with the aerosol of e-liquid or smoke of HNB cartridge, but the essence aromas can volatilize and be transmitted to the user's nasal cavity at room temperature, by slightly heating and or fan blowing, or with the assistance of the volatiles conduit, whereby maintaining the original flavor and physiological activity of the essence substances.

In addition, by providing a nasal inhalation module in the e-cigarette, and the nicotine is partially transferred or completely transferred from the mouth inhalation module into the nasal inhalation module of e-cigarettes, user's nasal cavity can directly inhale nicotine and quickly transfer signal to the brain, whereby improving the speed of nicotine satisfaction. Moreover, these nicotine molecules do not need to go through the step-by-step transmission process of many organs, which can be quickly volatilized out from the nasal inhalation module and be transferred to the user's nasal cavity at room temperature, by slightly heating and or fan blowing, or with the assistance of the volatiles conduit, resulting in high utilization rate of nicotine with low loss, which helps users to reduce the amount of nicotine intake each time for nicotine satisfaction by e-cigarettes.

Users can not only continue inhaling the atomized aerosol or HNB smoke by mouth which generated from the mouth inhalation module, but also directly inhale the essences, nicotine and other nasal inhalation material by nose which released from the nasal inhalation module of the e-cigarette, realizing dual inhalation channels to use the e-cigarette by cooperation of mouth and nose.

Twenty professional users of vapor e-cigarette evaluate the above-mentioned Comparative Example 1 and the Embodiments 1-5 respectively, and the comprehensive opinion shows that the employing experience of the e-cigarettes used by cooperation of mouth and nose in the Embodiments 1-5 is significantly better than that used by single mouth inhalation channel in the Comparative Example 1. Specifically, the evaluation is as follows: the Embodiments 1-5 enable the user's nasal cavity to perceive the essence aroma and nicotine more quickly, improving the transmission efficiency of essence molecules to the nasal cavity with the assistance of heating component and fan blowing in the nasal inhalation module especially in low temperature environment, and because there are not too much interference from other substances, the smell is more conducive to the rapid identification by the olfactory nerve, improving the flavor reproduction level of e-cigarette. In addition, the types of essence available for e-cigarettes are increased through the nasal inhalation module, even opaque and viscous tobacco extract paste, tobacco extract liquid, and tobacco smoke condensates can be well used in vapor e-cigarettes.

Twenty professional users of HNB e-cigarette compare the above-mentioned Comparative Example 2 with the Embodiments 6-7 respectively, and the comprehensive opinion is: although the cartridges of the Embodiments 6-7 contain a little or no essence and nicotine in the mouth inhalation module, after transferring the tobacco essence, tobacco extract liquid, and tobacco smoke condensates into the nasal inhalation module respectively, the employing experience of the HNB e-cigarettes used by cooperation of mouth and nose is better. The volatiles of essence and nicotine released from the nasal inhalation module can be perceived and identified by nose more quickly with the assistance of heating component and fan blowing in the nasal inhalation module especially in low temperature environments, whereby the physiological experience of the e-cigarette used by cooperation of mouth and nose is very close to that of traditional flue-cured tobacco.

In addition, the e-cigarette used by cooperation of mouth and nose in the present invention does not limit the inhaling material in the mouth inhalation module, wherein the mouth inhalation material may or may not contain nicotine and nicotine derivatives such as nicotine salt, and the likes, or it may contain cannabidiol, cannabis polyphenols or similar substances as the CBD factions electronic cigarette, and it may contain health-care substances such as tea polyphenols, licorice, lily or other herbal materials as a health device, and it may contain drug as a mouth suction-type medicinal instrument. The mouth inhalation material may be in one form of the gas, liquid, solid and paste.

In addition, the e-cigarette used by cooperation of mouth and nose in the present invention does not limit the working modes of the mouth inhalation module, wherein the mouth inhalation material may be heated by one of resistance heating, electromagnetic heating, infrared heating, microwave heating, as well as the mouth inhalation material being heated only a little when being pushed out as volatiles by some ultrasonic mode e-cigarette or devices. The mouth inhalation material can be heated not only by electrical energy but also by non-electrical energy and without using battery.

In addition, the e-cigarette used by cooperation of mouth and nose in the present invention does not limit the developing features of the mouth inhalation module, wherein the mouth inhalation module may adopts e-cigarettes or similar devices, such as high-power e-cigarette, low-power e-cigarette, multiple cartridges integrated e-cigarette, new appearance with some decoration, and others which is in or not in the prior art but inhaling material only by mouth.

In addition, the mouth inhalation module is an important part of the e-cigarettes used by cooperation of mouth and nose in the present invention, which not only remaining the material inhalation chancel and the sucking experience of mouth, but also some new functions may be developed for the e-cigarettes. For example: the inhalation nozzle module, which is one sub-module of the mouth inhalation module, inserts into user's mouth cavity each time, whereby some oral diseases may be sensed by touching the tongue, saliva or mouth mucus, as well as some messages of the intermediate organs may be captured from the residual aerosols or HNB smoke exhaled from the mouth and nose after the “small circulation through mouth” and “large circulation through lungs” by the mouth inhalation module. By analyzing the messages through the built-in sensors and analysis tools of the e-cigarettes to know whether the user is healthy and which chemical elements or material need to be supplied, such as iron, calcium, zinc, selenium, iodine, vitamin, and the likes, and the key data detected and important information may be communicated to the display module, whereby helping user to adjust the mouth inhalation material and the nasal inhalation material of the e-cigarettes by adding some medicinal materials or the needed chemical elements to improve his health correspondingly. The inhalation nozzle module in the mouth inhalation module may be further modified as an electronic tongue that can passionately kiss with the user, simulating a lover's tongue with similar shape and texture as the real tongue. When the electronic tongue touches with the user's tongue, it can further release some weak electrical waves or biological currents, making the user pleasure and leading the brain to secrete hormones such as dopamine, oxytocinand or adrenaline, as well as improving the enjoying of the e-cigarettes used by cooperation of mouth and nose of the present invention.

In addition, the e-cigarettes used by cooperation of mouth and nose in the present invention may develop some interesting functions. For example: the residual aerosols or HNB smoke, exhaled from the mouth and nose after the “small circulation through mouth” and “large circulation through lungs” by the mouth inhalation module, may be magnified without real material but by some artificial intelligence and or virtual and video projection technology in the display module to create a smoky scenery similar as or even beyond that of using traditional flue-cured tobacco or cigars, making the user pleasure to secrete dopamine in brain just because of eyes enjoying, whereby the e-cigarettes in the present invention can be used by cooperation of mouth, nose and eyes. Furtherly, the e-cigarettes used by cooperation of mouth and nose in the present invention may contain some components which can emit good music, sounds of nature, radio wave or joyful acoustic wave, such as Alpha wave, Beta wave, Gamma wave, Theta wave, and Delta wave, especially 40 hertz Gamma wave and or flashing stimuli may promote the secretion of dopamine in brain to replace the nicotine satisfaction, whereby the e-cigarettes in the present invention can be used by cooperation of mouth, nose, tongue, eyes and ears together and help user to further reduce the nicotine intake or even abandoning nicotine. Besides helping to secrete dopamine, the e-cigarettes in the present invention may be developed to help user's brain to secrete others pleasure hormones or beneficial emotions such as endorphins, oxytocin, phenylethylamine and serotonin, as well as helping user's body for curing diseases, treatment, rehabilitation, relaxation or sleeping when the e-cigarettes used by cooperation of more organs and corresponding components, with some substances being and or not being ingested into the body.

In addition, the e-cigarettes used by cooperation of mouth and nose in the present invention may integrate some electronic functions and or some human-machine interaction functions. For example: the display module can be arranged on the mouth inhalation module and or on the nasal inhalation module, which can be further developed as a touch control screen, a sound control screen, a virtual displays, a three-dimensional holographic projector, an artificial intelligence tool, a text and or language input device, a simultaneous translator, an intelligent answerer, a scheduling and reminders, a virtual reality and or augmented reality platform, an AI virtual assistant, and the likes.

In addition, the e-cigarettes used by cooperation of mouth and nose in the present invention may integrate many micro tools or objects. For example: USB data storage card, bank card, hazard perception alarm, legal micro self-defense weapon, flashlight, mobile phone with video projector, micro computer with virtual keyboard, handheld game console, camera, video recorder, wireless fidelity device, mobile network receiver, satellite communication equipment, carbon monoxide sensor, spray for sterilization and disinfection, mosquito killer, food poison detector, breath freshener, soap, perfume, incense, incense burner, mask, screwdriver, Swiss Army Knives, bottle opener, whistle or other musical device, megaphone, microphone, earphone, handwriting pen, note paper, laser pen, badge, chest tag, raincoat, rainproof shoe covers, plastic bag, emergency life tools, automatic inflation floating bag, robot, biochip, massager, condom, gloves, medicine bottle with drugs, chocolate, coffee bag, drinking water, straw that can filter and purify common water, lighter, traditional cigarettes, nicotine pouches, nicotine candy, chess, playing cards, retractable chopsticks, timers, inkpad, power bank, telescopes, microscopes, ropes, blind guider, mirror, cosmetics, drone, toothpick, and others micro tools or objects, may be partially or completely implanted inside the e-cigarettes set, whereby making the user's outside livings are very convenient.

In addition, the e-cigarettes used by cooperation of mouth and nose in the present invention may create a more big chamber space or more than one chamber spaces inside of not only the mouth inhalation module but also the nasal inhalation module by increasing the diameter of the e-cigarette rod or extending the shell more than 200 mm, and or enlarging size of the nasal inhalation module, whereby providing enough accommodation spaces for the above new functions and integrating new tools or objects as well as the original components of the e-cigarettes.

In addition, the e-cigarettes used by cooperation of mouth and nose in the present invention, the mouth inhalation module can be partially or completely implanted into the nasal inhalation module, which may be achieved by enlarging and modifying the nasal inhalation module or by other methods.

In addition, the e-cigarette used by cooperation of mouth and nose in the present invention may be integrated as one part of some tools or objects. For example: setting the e-cigarette outside or inside of a mobile phone and or the mobile phone case.

In addition, the e-cigarette used by cooperation of mouth and nose in the present invention can not only be produced and sold as a complete device containing both the mouth inhalation module and the nasal inhalation module, but also be produced and sold as some semi-finished product containing only the nasal inhalation module which may or may not integrate some of the above mentioned new functions and tools or objects, but the semi-finished product can be easily combined with the e-cigarettes or similar devices which inhaling material only by mouth through component retainers or other means to be a complete device that used by cooperation of mouth and nose. Then, the semi-finished product shall be included in the patent protection scope of the present invention. But the semi-finished product does not include the snuff bottle of the prior art, which only containing solid snuff powder and can not make the volatiles of the nasal inhalation material.

In addition, the e-cigarettes used by cooperation of mouth and nose in the present invention can has lock function or some biometric identification function in the above mentioned complete device and or in the semi-finished product, which may be unlocked or controlled by some mechanical buttons, digital passwords, iris recognition, facial recognition, fingerprint recognition, voice recognition, and the likes. These locks can prevent babies from misusing the e-cigarette, and some smart lock may further identify the user's age and prevent minors from using it.

In addition, the e-cigarettes used by cooperation of mouth and nose in the present invention can be miniaturized and or be streamlined, whereby having an intelligent wearable function in the above mentioned complete device and or in the semi-finished product, which may be as one part of a glasses, a wrist watch, a cap, a clothing, and the likes.

In addition, the e-cigarettes used by cooperation of mouth and nose in the present invention may partially or completely using some new materials. For example, the circuits are coated with hydrophobic materials to protect it from water damage; the nozzle is made of antibacterial material; the e-cigarette set are made of biodegradable plastic.

The using experience of the e-cigarettes used by cooperation of mouth and nose in the present invention may be more closer to or even beyond that of traditional flue-cured tobacco and cigars by reducing nicotine intake and reducing essence related dangerous substances entering the lungs, but improving the nicotine satisfaction and the secretion of dopamine and other pleasure hormones or beneficial emotions each time, as well as by developing the above new functions and integrating with many tools or objects.

The above description are only some embodiments of the present invention, and are not intended to limit the patent scope of the present invention. Any equivalent structure or equivalent process transformation made by using the content of the description of the present invention, and directly or indirectly applied in other related technical fields, shall be all included in the patent protection scope of the present invention.

1. An electronic cigarette used by cooperation of mouth and nose, wherein the electronic cigarette comprises a mouth inhalation module and a nasal inhalation module, the mouth inhalation module adopts one of a vapor electronic cigarette and a heat-not-burn electronic cigarette, the nasal inhalation module is arranged outside or inside of the mouth inhalation module, and the nasal inhalation module contains at least one nasal inhalation material carrier, and a nasal inhalation material is placed in the nasal inhalation material carrier, the nasal inhalation module further contains a component that can heat the nasal inhalation material, a first battery is arranged in the mouth inhalation module, and the nasal inhalation module shares the first battery with the mouth inhalation module, or the nasal inhalation module contains a second battery or without battery.

2. The electronic cigarette used by cooperation of mouth and nose according to claim 1, wherein the vapor electronic cigarette adopts one of a CBD factions electronic cigarette, an open type electronic cigarette, a closed type electronic cigarette, and the heat-not-burn electronic cigarette adopts one of a heat-not-burn electronic cigarette that consuming solid cartridge and a heat-not-burn electronic cigarette that consuming paste material.

3. The electronic cigarette used by cooperation of mouth and nose according to claim 1, wherein the nasal inhalation module is arranged on the shell of the mouth inhalation module.

4. The electronic cigarette used by cooperation of mouth and nose according to claim 1, wherein a chamber corresponding to the nasal inhalation module is arranged inside of the mouth inhalation module, and the nasal inhalation module is partially or completely implanted into the chamber.

5. The electronic cigarette used by cooperation of mouth and nose according to claim 1, wherein the electronic cigarette further comprises a component retainer for fixing the nasal inhalation module outside or inside of the mouth inhalation module, the component retainer adopts one or more of double-sided adhesive tape, gummed tape, glue, string, clamp, snap hook, magnet, screw, slot, jack and plug, and fixing ring.

6. The electronic cigarette used by cooperation of mouth and nose according to claim 1, wherein the nasal inhalation material carrier adopts one or more of a bottle, a pot, an electric heating plate and an electric heating pot, and the electric heating plate adopts a miniature mesh electric heating plate or a non-mesh electric heating plate with integrated heating component.

7. The electronic cigarette used by cooperation of mouth and nose according to claim 1, wherein the nasal inhalation material is a combination of one or more of essence, alkaloid, nicotine salt, e-liquid, tobacco extract paste, tobacco extract liquid, tobacco smoke condensate, cannabinoids, essence solvents, but air does not belong to the nasal inhalation material, and the essence is one or more of fruit peel, fruit, fruit essence, cooling agent essence, sweetener essence, tobacco essence, meat essence, wine essence, food essence, plant herbal essence, animal spice essence, biosynthetic essence and microbial fermentation essence.

8. The electronic cigarette used by cooperation of mouth and nose according to claim 1, wherein the nasal inhalation module further contains an electric heater controller, and the first battery or the second battery is connected with the electric heater controller for power supply, and the electric heater controller is connected with the heating component for heating control in the nasal inhalation module.

9. The electronic cigarette used by cooperation of mouth and nose according to claim 1, wherein the component that can heat the nasal inhalation material comprises a combination of one or more of battery, circuit switch, controller, wire, heater.

10. The electronic cigarette used by cooperation of mouth and nose according to claim 1, wherein the nasal inhalation module further comprises a fan and a fan controller corresponding to the nasal inhalation material, and the first battery or the second battery is connected with the fan controller for power supply, and the fan controller is connected with the fan for operation control.

11. The electronic cigarette used by cooperation of mouth and nose according to claim 1, wherein the nasal inhalation module further comprises a volatiles conduit, and one end of the volatiles conduit is close to the nasal inhalation material carrier or directly or indirectly connected with the nasal inhalation material carrier.
